(19) United States
(12) Patent Application Publication
(10) Pub. No.: US 2012/0114578 A1
(43) Pub. Date: May 10, 2012

(54) Paste Composition For Use In Coating Films, Fibers, And Fabrics And Method of Manufacture

(75) Inventors: JOHN H. RICHARDS, III, Sylvania, OH (US); VERONICA A. COLEMAN, Tecumseh, MI (US); MICHAEL L. COFFEY, Adrian, MI (US)

(73) Assignee: WACKER CHEMICAL CORPORATION, Adrian, MI (US)

(21) Appl. No.: 12/939,611

(22) Filed: Nov. 4, 2010

(51) Int. Cl. A61K 8/92; A61F 13/15; B32B 27/04; C10M 107/50; A61Q 15/00; B32B 27/00

(52) U.S. Cl. 424/65; 442/99; 428/391; 508/207; 508/161; 604/367

(57) ABSTRACT

A paste composition for use in coating film, fiber, or fabric includes a dry grease having a discontinuous aqueous phase having a non-ionic alkoxylate surfactant portion and a polyorganosiloxane oil phase. The past further includes a dry-grease suspended powder. The surfactant portion is effective to suspend the powder forming a self-supporting form.

### BACKGROUND

1. Technical Field

The present invention relates to a paste composition for use in coating film, fiber, and fabric, and a method of manufacture.

2. Background Art

Fabrics, fibers, and films are used in many circumstances to provide a carrier for a coating. Silicone oils have been used to act as a carrier vehicle and a lubricious coating. However, when powdered solids, such as talc and cornstarch, are added to many liquids, such as silicone oil, the powdered solids do not remain in suspension or solution and accumulate at the bottom of a solution containing them.

### SUMMARY

One embodiment of the present invention comprises a paste composition for use in coating film, fiber, or fabric that includes a dry grease. The dry grease comprises a discontinuous aqueous phase having a non-ionic alkoxylate surfactant portion and a polyorganosiloxane oil phase. The paste further includes a dry-grease-suspended powder. The surfactant portion in the polyorganosiloxane oil portion when present in the dry grease in an effective amount to suspend the powder, forms a self-supporting form.

In another embodiment, a paste composition includes a filled silicone-containing composite comprising a substantially anhydrous water-in-oil emulsion. The emulsion includes a surfactant, a film-forming silicone oil having a viscosity ranging from 5 centipoise to 60,000 centipoise, and a powder. The paste further includes water in an effective amount to form a coatable, filled, silicone-containing composite.

In yet another embodiment of a method of manufacture of a paste for use in coating film, fiber, and fabric includes the step of providing a blend of water and a surfactant. The ratio of water to surfactant ranges from 1 to 3 by weight of the blend. A silicone oil is sheared into the blend to form a self-supporting water-in-oil form. The silicone oil comprises an amount ranging from 50% by weight to 85% by weight of the form. The form is self-supporting for a time period after shearing ceases. A solid is mixed with the self-supporting water-in-oil form to form a first paste. The solid comprises 4% by weight to 30% by weight of the paste composition.

In yet another embodiment an article comprising a substrate selected from a group consisting of a woven fabric, a non-woven fabric, a fiber, and a film is recited. The article includes a paste having a dry grease including a powder disposed therein. The paste is disposed adjacent to or incorporated into the substrate. The dry grease comprises a water-in-oil emulsion. The emulsion comprises a non-ionic alkoxylate surfactant, water, and a polyorganosiloxane oil. The surfactant has a ratio of molar content of alkylate content to alcohol content ranging from 4 to 10.

### BRIEF DESCRIPTION OF THE DRAWINGS

FIG. 1 schematically illustrates a fragmentary cross-sectional view of a coated substrate according to at least one embodiment;

FIG. 2 schematically illustrates a cross-sectional view of a coated fiber according to at least one embodiment; and

FIG. 3 diagrammatically illustrates a method of manufacture of a paste according to at least one embodiment.

### DETAILED DESCRIPTION OF THE EMBODIMENTS

Detailed embodiments of the present invention are disclosed herein; however, it is to be understood that the disclosed embodiments are merely exemplary of the invention that may be embodied in various and alternative forms. The figures are not necessarily drawn to scale, some features may be exaggerated or minimized to show details of particular components. Therefore, specific structural and functional details disclosed herein are not to be interpreted as limiting, but merely as a representative basis for the claims and/or as a representative basis for teaching one skilled in the art to variously employ the present invention.

Except in the operating examples, or where otherwise expressly indicated, all numbers in this description indicating material amounts, reaction conditions, or uses are to be understood as modified by the word “about” in describing the invention's broadest scope. Practice within the numerical limits stated is generally preferred. Also, unless expressly stated to the contrary:
percent and ratio values are by weight;
the term “polymer” includes “oligomer,” “copolymer,” “dimer,” “terpolymer,” “tetramer” and the like;
a material group or class described as suitable or preferred for a given purpose in connection with the invention implies any two or more of these materials may be mixed and be equally suitable or preferred;
constituents described in chemical terms refer to the constituents at the time of addition to any combination specified in the description, and does not preclude chemical interactions among mixture constituents once mixed;
an acronym's first definition or other abbreviation applies to all subsequent uses here of the same abbreviation and mutatis mutandis to normal grammatical variations of the initially defined abbreviation; and
unless expressly stated to the contrary, measurement of a property is determined by the same technique as previously or later referenced for the same property.

In at least one embodiment, a relatively dry coating provides a carrier vehicle for powdered solids, adapting them to be suitable for application to fabrics, films, and fibers. Further, the dry coating is suitable for materials in contact with human skin and other living, sensitive surfaces. A non-limiting example of the coating is a lubricious coating.

In order to form the dry coating, a paste composition is formed from a meringue, which is converted to a dry grease. A powder is suspended in the dry grease in order to provide a paste for applying a lubricious coating. In certain embodiments, the lubricious coating has relatively good moisture absorption properties. When using such paste, portions of the dry grease, which may act as a carrier vehicle, flash off leaving a solid, relatively dry coating on a film, a fiber, or a fabric. In certain embodiments, the dry coating is dry to the touch. Also, in certain embodiments, when the dry coating is wiped off of a substrate, the friction coefficient of a surface of the substrate is lower than before the dry grease was first applied to the surface.

The meringue comprises a blend of a surfactant and water when in a low-shear mixer. The meringue has a density, in at least one embodiment, ranging from 0.7 to 0.9. In another embodiment, the meringue has a density ranging from 0.74 to 0.83.

A non-limiting example of the dry grease is a mixture of a dry-set lubricant which is delivered with a carrier vehicle including the meringue. An example of a carrier vehicle may be one or more solvents that evaporate to leave the dry-set lubricant as a film. In many embodiments, the dry-set lubricant attracts relatively little, if any, dust from the environment. Examples of dry-set lubricants include silicones and polytetrafluoroethylene (PTFE).

In at least one embodiment, dry grease may be comprised of an anhydrous water-in-oil emulsion. A non-limiting example of the anhydrous water-in-oil emulsion may comprise the surfactant and water, such as the meringue and a film-forming silicone oil. In at least one embodiment, the water comprises less than 10% by weight of the anhydrous water-in-oil emulsion by weight. In another embodiment, the anhydrous water-in-oil emulsion may include water comprising a range from 0.1 wt. % to 8 wt. %. In yet another embodiment, the anhydrous water-in-oil emulsion may include water ranging from 3 wt. % to 7 wt. %. In at least one embodiment, the dry grease has a discontinuous aqueous phase.

The dry grease comprises a low-shear blend of the oil phase and the aqueous phase. In at least one embodiment, the low-shear blend comprises a blend having a normal force of less than 10,000 reciprocal seconds. In another embodiment of the invention, the low-shear blend comprises a blend having a normal force ranging from 1,000 reciprocal seconds to 9,000 reciprocal seconds. In yet another embodiment of the invention, the low-shear blend comprises a blend having a normal force ranging from 4,000 reciprocal seconds to 8,000 reciprocal seconds. In at least one embodiment, the low-shear blend is generated by a Hobart® mixer using a low shear speed at an intermediate setting of less than 500 rpm for an agitator or an impeller and/or 200 rpm for an attachment. In another embodiment, the low speed is less than or equal to 136 revolutions per minute (rpm) for the agitator and less than 60 rpm for the agitator.

In at least one embodiment, the dry grease has a viscosity ranging from 1 million to 1.8 million centipiose when measured using a paste viscometer. In at least another embodiment, the dry grease has a viscosity ranging from 1.1 to 1.6 million centipiose. The paste viscometer includes a T-bar spindle attached to the viscometer where the drive motor of the viscometer slowly lowers and raises the viscometer relative to the T-bar spindle to create a helical path through the test sample to eliminate channeling as an artifact of the measurement method. A non-limiting example of a paste viscometer is a Brookfield® viscometer operated in the RV viscosity range, HA viscosity range, or HB viscosity range.

In at least one embodiment, the dry grease has a density range from 0.9 to 1. In another embodiment, the dry grease has a density ranging from 0.92 to 0.96.

It should be understood that additional water may be added in a subsequent step such that without exceeding the scope of the invention the water content of a mixture of the anhydrous water-in-oil emulsion and the added water comprises more than 10% by weight of the mixture. It should be understood that when the added water comprises more than 10% by weight of the mixture, the mixture may become a water-in-oil emulsion and/or an oil-in-water emulsion. The water-in-oil emulsion and oil-in-water emulsion, it should be understood, do not exceed the contemplated scope of embodiments.

The surfactant in the meringue used to form the dry grease is soluble in the water and may function to organize the film-forming silicone oil phase. In at least one embodiment, the surfactant has a hydrophilic to lipophilic balance (HLB) ranging from 7 to 16 when measured using the Griffin method. In another embodiment, the surfactant may have the HLB ranging from 9 to 14. In yet another embodiment of the present invention, the surfactant may have the HLB ranging from 11 to 12.5.

The hydrophilic to lipophilic balance of the surfactant, as determined by the Griffin method, assists in the selection of an appropriate surfactant from the staggering number of surfactants that are available. Those skilled in the art may appreciate that there is a correlation between the surfactant's behavior and its solubility in water. The relationship of the surfactant's behavior and its water solubility may be suggested by the ratio of a weight of the hydrophilic groups in the surfactant molecule to a weight of the lipophilic groups in the surfactant molecule when non-ionic surfactants are used. The HLB value, as determined by the Griffin method, may be understood to be a function of the weight percentage of the hydrophilic portion of the non-ionic surfactant molecule. It should be understood that HLB values may be calculated for non-ionic surfactants or may be determined experimentally.

In surfactants wherein only alkylene oxide is used as the hydrophilic portion and/or fatty alcohol alkylene oxide condensation products, the HLB equation, according to the Griffin method, may be simplified to:

$$H L B = \frac{wt . \% of alkylene content}{5}$$

In the more general case, Griffin's method for non-ionic surfactants uses the equation:

$$H L B = 20 \times \frac{\begin{matrix}molecular mass of the hydrophlic \\ portion of the surfactant molecule\end{matrix}}{the total molecular mass of the surfactant molecule}$$

The HLB value can be used to predict the surfactant properties of the molecule, such as follows:

an HLB value ranging from 0 to 3 indicates an anti-foaming agent,

an HLB value from 4 to 6 indicates a water-in-oil emulsifier,

an HLB value ranging from 7 to 9 indicates a wetting agent,

an HLB value ranging from 8 to 18 indicates an oil-in-water emulsifier,

an HLB value ranging from 13 to 15 indicates a detergent, and

an HLB value ranging from 10 to 18 indicates a solubilizer or a hydrotrope.

It is surprising, then, that the dry grease from the meringue using the surfactant used in certain embodiments would exhibit the HLB values more typical of wetting agents, oil-in-water emulsifiers, and detergents while having a composition of the anhydrous water-in-oil emulsion. In at least one embodiment of the present invention, the HLB for the dry grease within any temperature constraints may range from 7.9 to 16. In at least one other embodiment of the present invention, the dry grease has a HLB ranging from 9 to 14. In yet another embodiment of the present invention, the dry grease has an HLB ranging from 11 to 12.5.

In at least one embodiment, the surfactant may include a long-chain alcohol polyether, such as a fatty alcohol polyether. In another embodiment, the surfactant may include an alkoxylated fatty alcohol having a carbon chain ranging from C₁₀ to C₃₀. In yet another embodiment of the present invention, the surfactant may have an alkoxylated fatty acid having a carbon chain ranging from C₁₀ to C₁₈. In yet another embodiment of the present invention, the alkoxylated fatty alcohol may have a carbon chain ranging from C₁₂ to C₁₄. The surfactant may also include a non-ionic composition such as tridecyl alcohol ethoxylate (C₁₃H₂₇)(OCH₂CH₂)ₙOH. (CAS 24938-91-8).

It should be understood that while primary aliphatic alcohol polyethers, such as fatty alcohol polyethers are disclosed, in yet other embodiments, β-alkylated dimer alcohol polyethers, such as Guerbet alcohol polyethers, may be used without exceeding the scope of embodiments contemplated herein.

In at least one embodiment, the alkoxylated fatty alcohol has a ratio of an alkoxylate content to the alcohol content ranging from 4 to 10 molar ratio. In another embodiment, the ratio of the alkoxylated fatty alcohol alkoxylate content to the alcohol content ranges from 6 to 9 molar ratio. In yet another embodiment, the ratio of the alkoxylated fatty alcohol alkoxylate content to the alcohol content ranges from 7 to 8.75 molar ratio.

In at least one embodiment, the alkoxylated fatty alcohol may comprise a polyoxyalkene fatty alcohol. Non-limiting examples of polyoxyalkene groups on the fatty alcohol may include polyoxyethylene groups, polyoxypropylene groups, and combinations thereof.

The amount of surfactant may range from 5 wt. % to 20 wt. % of the dry grease. In another embodiment, the amount of surfactant may range from 8 wt. % to 13 wt. % of the dry grease.

In at least one embodiment, the film-forming silicone oil may include a dimethicone. Non-limiting examples of the dimethicone may include a polyorganosiloxane, a polyalkylsiloxane, an alkylmethylsiloxane, a silicone glycol copolymer, an amino functional silicone, and/or a cyclosiloxane.

A non-limiting example of the dimethicone is polydimethylsiloxane, such as a linear, non-reactive unmodified polydimethylsilane, which has a viscosity ranging from 5 centipoise to 60,000 centipoise in at least one embodiment. In another embodiment, the polydimethylsiloxane has a viscosity ranging from 10 to 7000 centipoise. In yet another embodiment of the present invention, the polydimethylsiloxane has a viscosity ranging from 90 to 1500 centipoise.

In at least one embodiment, the dimethicone has a relatively high permeability to gases, such as water vapor and oxygen, suitable for allowing respiration of moisture from the skin. In one embodiment of the invention, the water vapor permeability ranges from 100 gm/m²/hr to 120 gm/m²/hr. In another embodiment, the dimethicone has a water vapor permeability ranging from 110 gm/m²/hr to 119 gm/m²/hr. In yet another embodiment, the dimethicone has a water vapor permeability ranging from 114 gm/m²/hr to 118.5 gm/m²/hr.

In at least one embodiment of the invention, the dimethicone is a substantially non-volatile fluid. The volatility of the fluid may be influenced by the chain length of the alkyl substituent disposed at the ends of the siloxane polymer backbone. In at least one embodiment, the length of the substituent ranges from C₁ to C₁₈. In another embodiment, the substituent may range from C₁ to C₁₂. It should be understood that the substituents at either end of the siloxane polymer backbone may be the same or different.

In at least one embodiment, the dimethicone has a % volatiles content less than 15 wt. %. In another embodiment, the dimethicone has a % volatiles content less than 7 wt. %.

The film-forming silicone oil may have a relatively low surface tension and a relatively high spreading coefficient. In at least one embodiment of the present invention, the film-forming silicone oil has a surface tension at 25° C. ranging from 15 mN/m to 25 mN/m. In another embodiment, the film-forming silicone oil has a surface tension ranging from 19 mN/m to 24 mN/m.

The amount of film-forming silicone ranges, in at least one embodiment, from 40 wt. % to 95 wt. % of the paste. In another embodiment, the amount of film-forming silicone ranges from 50 wt. % to 70 wt. % of the paste. The amount of silicon in the paste ranges, in at least one embodiment, from 12 wt. % of silicon in the paste to 35 wt. % of silicon.

The amount of silicone in the dry grease ranges from 48 wt. % of the dry grease to 99 wt. % of the dry grease. In another embodiment, the amount of silicone in the dry grease ranges from 60 wt. % to 90 wt. %.

In at least one embodiment, the film-forming silicone leaves a substantially continuous layer over all of the powder in the dry grease and the paste. In another embodiment, the film-forming silicone coalesces when laid on a powder above a minimum film-forming temperature as measured by ISO 2115.

The powder suspended in the dry grease may include a hydrophilic powder, a hydrophobic powder, a powder with relatively low hydrophilicity, a moisture-absorbing powder, a lubricating powder, and combinations thereof. The powder may be a moisture absorbing powder or a lubricating powder. The carrier vehicle, such as the dry grease, the film-forming silicone, and/or the anhydrous water-in-oil emulsion may not cause substantial wetting of the powder in order that the powder can function as a liquid-absorbing agent on the film, fiber, or fabric.

In certain embodiments, the wetting of the powder by the dry grease surfactant, water, and/or silicone oil is minimized. In at least one embodiment, the powder has a maximum moisture content prior to suspension of less than 2 wt. % when measured by ASTM D 605. In another embodiment, the powder has a maximum moisture content prior to suspension of less than 1 wt. %.

In at least one embodiment, the shape of the powder may be relatively platy, such as a talc. In another embodiment of the present invention, the powder may have a relatively spherical shape, such as a polysaccaride composition including a starch or a modified starch.

The shape of the powder may be measured by scanning electron microscopy of between 100 and 200 particles. The particles may assessed by measuring the length to width ratio to determine an average length to width ratio.

While not wishing to be constrained by any particular theory, in certain embodiments the more plate-like the powder, the more readily the powder bridges the carrier vehicle polymer molecules. The more bridging that occurs may increase the likelihood of forming or sustaining a self-supporting form with a relatively reduced amount of powder. In at least one embodiment, the powder has a shape having the average length to width ratio ranging from 1 to 10. In another embodiment, the average length to width ratio ranges from 1.1 to 8. In another embodiment, the average length to width ratio ranges from 2 to 7.

In at least one embodiment, the powder has an average particle size ranging in diameter of a maximum axis ranging from 5 μm to 10 μm. In another embodiment, the powder has an average particle size ranging in diameter of a maximum axis from 6 μm-8 μm.

It should be understood that the powder in certain embodiments may include a surface treatment to modify the compatibility with the dry grease and still be within the scope of the embodiments contemplated herein.

In at least one embodiment, the powder may comprise, but is not limited to, an astringent, a soap, a silicate, a metal salt, a metal acid, and/or a hydrophilic organic material.

The powder suspended in the dry grease comprises a range of 4% by weight to 30% by weight as dry solids of the paste in at least one embodiment. In another embodiment, the powder suspended in the dry grease comprises a range from 8% by weight to 20% by weight as dry solids of the paste. In yet another embodiment of the invention, the powder suspended in the dry grease comprises a range from 10% by weight to 18% by weight as dry solids of the paste.

The paste composition, in at least one embodiment, forms a self-supporting form having a Voland hardness ranging from 50 gm to 110 gm when measured according to test method ASTM D-638 when measured with a Texture Analyzer (Brookfield®, model LFRA) with a 0.5″ spherical probe. In at least another embodiment, the paste composition has a self-supporting form with a Voland hardness ranging from 70 gm to 105 gm. In yet another embodiment of the invention, the composition has a self-supporting form with a Voland hardness ranging from 75 gm to 100 gm.

The paste, in at least one embodiment, has a viscosity ranging from 300,000 centipoise to 1,500,000 centipoise. In another embodiment, the paste has a viscosity ranging from 700,000 centipoise to 1,100,000 centipoise.

It should be understood that dry grease, paste, and meringue are embodiments of dispersions.

FIG. 1 schematically illustrates a coated substrate 10 having the paste 12 on a substrate 14 according to at least one embodiment. Paste 12 has powder 16 suspended in a matrix 18 comprising the surfactant, water, and silicone. Substrate 14, in at least one embodiment, is a film, such as a thermoplastic sheet or a polyolefin layer. In another embodiment, substrate 14 comprises a fabric such as, but not limited to, a web, a spun-bonded layer, a woven fabric, a non-woven fabric, and/or a lawn. Non-limiting examples include a personal care article, a household care article, a tissue paper, a diaper, a skin wipe, a deodorant solid, and/or a skin-engaging film. Paste 12 may be applied to substrate 14 by methods known in the art such as, but not limited to, spraying, screeting, and rolling.

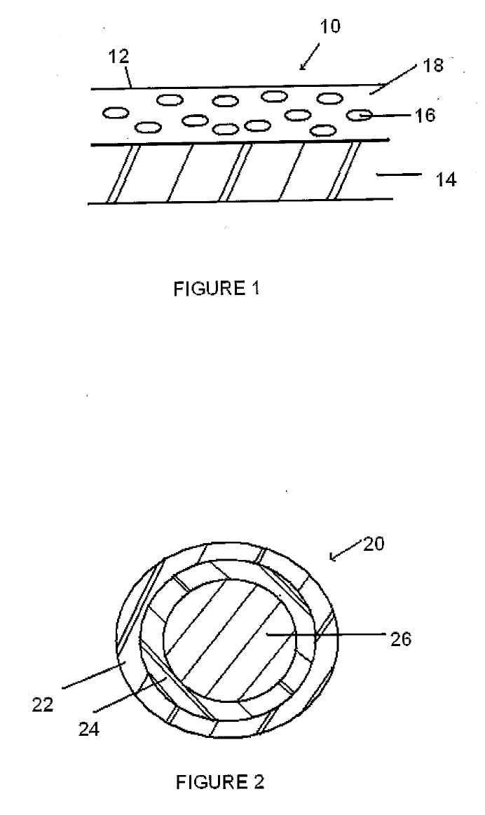

FIG. 2 schematically illustrates a cross-sectional view of a coated fiber 20 having a fiber 26 covered with a plurality of layers 22 and 24 of paste according to at least one embodiment. Non-limiting examples of the fiber 26 include an oriented fiber, a multi-filament fiber, a monofilament fiber, a synthetic strand, a natural material strand, and/or a netting woven from fibers. It is understood that layers 22 and 24 may comprise the same or different compositions.

Paste 12, with or without the second addition of water, may be applied to fiber 26 by methods known in the art, such as dipping, spraying, or vacuum painting.

Paste 12 may be applied in at least one embodiment to substrate 12 or fiber 26 in an amount ranging from 10 gm/m² to 100 gm/m². In another embodiment, paste 12 may be applied to substrate 14 or fiber 26 in an amount ranging from 15 gm/m² to 50 gm/m². In yet another embodiment, paste 12 may be applied to substrate 14 effective to prevent a liquid from passing through substrate 14.

In yet another embodiment, paste 12 may be applied to substrate 14 and/or fiber 26 such that the coated substrate 10 and/or coated fiber 20 have a water vapor transmission rate ranging from 1000 gm/m²/24 hr. to 3000 gm/m²/24 hr. when measured using 2 μL/cm² layer applied to a collagen film placed on a permeability cup at 24° C. and 45% relative humidity.

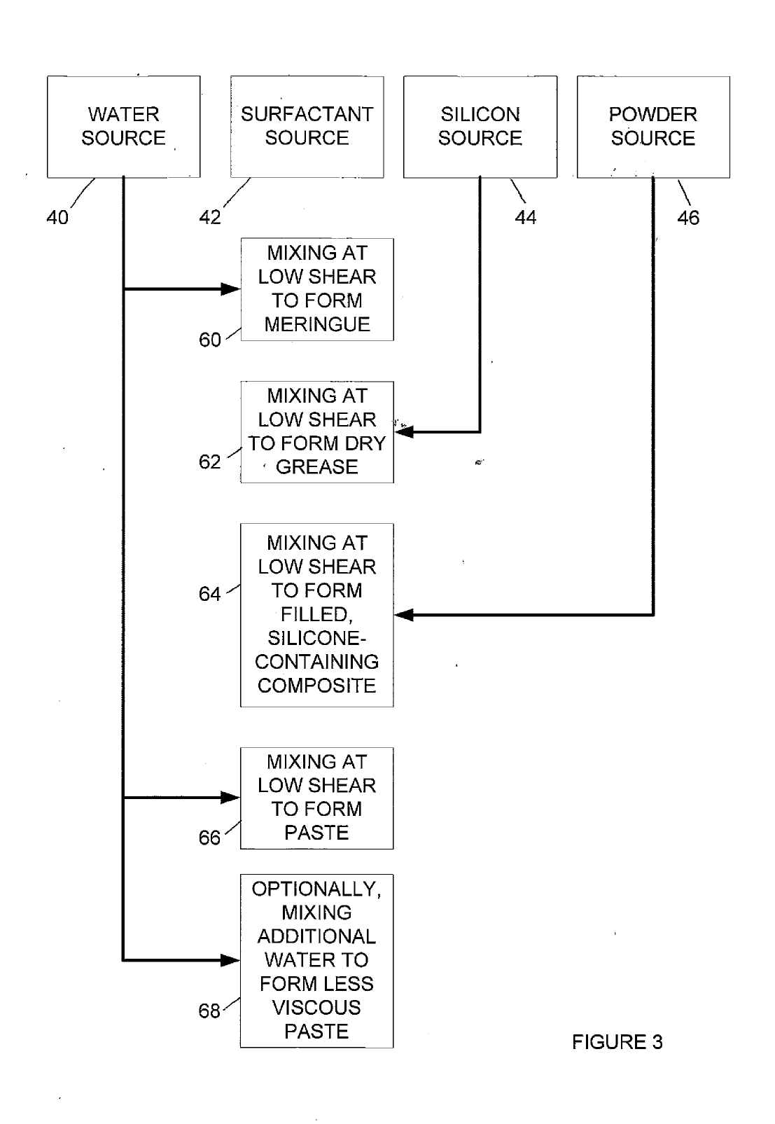

Turning now to FIG. 3, one embodiment of a method of manufacture of paste 12 is diagrammatically illustrated. In step 60, one-third to one-half of the deionized water portion is dispensed from a deionized water source 40 into a mixer. Surfactant is dispensed from a surfactant source 42 into the mixer. In at least one embodiment, the ratio of water to surfactant ranges from 1 to 3 by weight of the blend. In step 60, the meringue is formed by low shear mixing of deionized water and surfactant.

In step 62, the silicone oil is dispensed from a silicone source 44 into the mixer and mixed at the low shear rate to form the dry grease, such as the anhydrous-oil-in-water emulsion, which in certain embodiments is a self-supporting water-in-oil emulsion.

In step 64, the powder is dispensed from a powder source 46 into the mixer and mixed at the low shear rate to form the powder suspended in dry grease forming a filled, silicone-containing composite.

In step 66, the powder in suspended dry grease receives one-half to two-thirds of the deionized water portion from the deionized water source 40. Mixing at the low shear rate continues until the paste, such as the water-in-oil emulsion, is formed. The paste may comprise a coatable, filled, silicone-containing composite.

The paste may be removed from the mixer and applied to fabric, film, and/or fiber by methods described above.

Optionally, in step 68, additional fluid, such as water or an alcohol, may be selected to adjust the paste viscosity or other physical property, to form a second paste.

In certain embodiments, the paste comprises a non-Newtonian fluid. In other embodiments, the paste comprises a substantially Newtonian fluid. It is understood that addition of fluid to form a second paste may induce inversion of the water-in-oil emulsion to the oil-in-water emulsion.

### EXAMPLES

### Example 1

The paste is prepared as follows in Table 1:

TABLE 1

| | |
|---|---|
| Deionized water | 15.69 wt. % |
| Surfactant^{a} | 10.72 wt. % |
| Film-forming silicone^{b} | 65.26 wt. % |
| Powder^{c} | 8.33 wt. % |

^{a}Dethox TDA 8.5 supplied by Deforest Enterprises, Inc. (Florida)
^{b}DM 1000 Fluid supplied by Wacker Chemical Corp. (Michigan)
^{c}Talc supplied by Sensient Technologies (New Jersey)

The paste is prepared by placing less than half of the water in a Hobart mixer at the intermediate Hobart setting.

The surfactant is added slowly over a time period exceeding two minutes to form the meringue.

To the meringue is added the film-forming silicone at the intermediate Hobart setting to form the dry grease.

The talc is sifted into the dry grease over a time period exceeding two minutes at the low shear condition of the intermediate Hobart setting.

The second portion of water is slowly added to the powder and dry grease at the low shear condition of the intermediate Hobart setting to form the paste.

### Example 2

The paste is prepared as follows in Table 2:

TABLE 2

| | |
|---|---|
| Deionized water | 21.15 wt. % |
| Surfactant | 9.12 wt. % |
| Film-forming silicone | 55.55 wt. % |
| Powder^{a} | 14.18 wt. % |

^{a}Cornstarch supplied by Corn Products (Illinois)

The paste is prepared by placing less than a third of water in a Hobart mixer at the lower shear condition of the intermediate setting.

To the meringue is slowly added the film-forming silicone at the intermediate setting to form a dry grease. The addition occurs over a time period exceeding two minutes.

The cornstarch powder is slowly sifted into the dry grease. The Hobart mixer is used at the intermediate setting. The sifting occurs over a time period exceeding two minutes.

The powder and dry grease receive the balance of the water slowly over a time period exceeding two minutes. The Hobart mixer is used at the intermediate setting.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A paste composition for use in coating films, fibers, and fabrics, the composition comprising:
a dry grease comprising a discontinuous aqueous phase having a non-ionic alkoxylate surfactant, and a polyorganosiloxane oil phase; and
a powder being suspended in the dry grease, wherein the dry grease has an effective quantity of surfactant portion and an effective amount of the polyorganosiloxane oil to form a self-supporting form.

2. The composition of claim 1, wherein the dry grease has a hydrophilic-lipophilic balance in the range from 7.9 to 16 when measured according to the Griffin method.

3. The composition of claim 1, wherein the paste has a polyorganosiloxane content ranging from 40% by weight to 95% by weight on a solids basis of the paste.

4. The composition of claim 1, wherein the powder comprises at least one of a moisture absorbing powder or a lubricating powder.

5. The composition of claim 1, wherein the powder is present and comprises a polysaccharide composition.

6. The composition of claim 4, wherein the powder is present and comprises a talc composition.

7. The composition of claim 1, wherein the powder comprises a range from 4% by weight to 30% by weight as dry solids of the paste.

8. The composition of claim 8, wherein the dry grease comprises a low-shear blend of the oil phase and the aqueous phase, wherein the aqueous phase comprises less than 10% weight of the dry grease.

9. The composition of claim 1, wherein the shape of the powder has an average length to width ratio ranging from 1 to 10.

10. The composition of claim 1, wherein the dry grease has a viscosity ranging from 1 million to 1.8 million centipoise when measured using a paste viscometer.

11. The composition of claim 1, wherein the self-supporting form has a Voland hardness ranging from 50-110 gm when measured according to test method ASTM D-638.

12. A paste composition for use in coating films, fibers, and fabrics, the composition comprising:
a filled silicone-containing composite comprising a substantially anhydrous water-in-oil emulsion including a surfactant, a film-forming silicone oil having a viscosity ranging from 5 centipoise to 60,000 centipoise, and a powder; and
water, wherein the water is in an effective amount to form a coatable, filled, silicone-containing composite.

13. The composition of claim 12, wherein the surfactant comprises an alkoxylated fatty alcohol having a carbon chain ranging from C₁₀ to C₃₀.

14. The composition of claim 12, wherein the substantially anhydrous water-in-oil emulsion has a water content of less than 10 wt % of the filled silicone-containing composite.

15. The composition of claim 12, wherein the silicone oil is selected from a group consisting of a polyalkylsiloxane, a polyarylsiloxane, a polycyclosiloxane, an aminofunctional siloxane, a silanol, a branched silicone, and a linear silicone.

16. The composition of claim 12, wherein the water comprises a range from 10 wt % to 30 wt % of the paste.

17. The composition of claim 12, wherein the coatable filled silicone-containing composite has a viscosity ranging from 300,000 centipoise to 1,500,000 centipoise.

18. The composition of claim 12, wherein the powder is selected from a group consisting of an astringent, a soap, a silicate, a metal salt, a metal acid, and a hydrophilic organic material.

19. A method of manufacture of a paste composition for use in coating films, fibers, and fabrics, the method comprising the steps of:
(a) providing a blend of water and a surfactant, the ratio of water to surfactant ranging from 1 to 3 by weight of the blend;
(b) shearing a silicone oil into the blend to form a self-supporting water-in-oil emulsion, the silicone oil comprising a range of 50 wt % to 85 wt % of the emulsion, the emulsion being self-supporting for a time period after shearing ceases; and
(c) mixing a solid with the self-supporting water-in-oil emulsion to form a first paste; the solid comprising 4% by weight to 30% by weight on a dry solids basis of the paste composition.

20. The method of claim 19, further comprising the step of reducing the viscosity of the first paste with a liquid additive to form a second paste.

21. The method of claim 20, wherein the second paste comprises a non-Newtonian fluid.

22. An article comprising:
a substrate selected from the group consisting of a woven fabric, a non woven fabric, a fiber, and a film; and
a paste including a dry grease having a powder disposed therein, the paste being disposed adjacent to or incorporated into the substrate, the dry grease comprising a water-in-oil emulsion comprising a non-ionic alkoxylate surfactant, water, and a polyorganosiloxane oil, wherein the surfactant comprises a ratio of molar content of alkylate content to alcohol content ranging from 4 to 10.

23. The article of claim 22, wherein the powder comprises 4% by weight to 30% by weight on a dry solids basis of the paste composition.

24. The article of claim 22, wherein the article is selected from the group consisting of a personal care article, a household care article, and a tissue paper.

25. The article of claim 24, wherein the personal care article is selected from a group consisting of a diaper, a skin wipe, a deodorant solid, and a skin engaging film.
